(19) United States
(12) Patent Application Publication
(10) Pub. No.: US 2006/0289309 A1
(43) Pub. Date: Dec. 28, 2006

(54) Microchemical system

(75) Inventors: Takashi Fukuzawa, Tokyo (JP); Jun Yamaguchi, Tokyo (JP); Kenji Uchiyama, Tokyo (JP); Akihiko Hattori, Tokyo (JP)

Correspondence Address: COHEN, PONTANI, LIEBERMAN & PAVANE, 551 FIFTH AVENUE, SUITE 1210, NEW YORK, NY, 10176, US

(73) Assignee: Nippon Sheet Glass Company, Limited, Tokyo (JP)

(21) Appl. No.: 11/513,680

(22) Filed: Aug. 30, 2006

Related U.S. Application Data
(63) Continuation of application No. PCT/JP05/02507, filed on Feb. 10, 2005.

(30) Foreign Application Priority Data
Mar. 3, 2004 (JP) 2004-058957 (PAT.

(51) Int. Cl. C07K 1/26

(52) U.S. Cl. 204/451

(57) ABSTRACT

A microchemical system is disclosed that is capable of controlling the flow of a sample solution flowing through a channel in a microchip. In the microchemical system 1, a microchip 7 has therein a T-shaped channel 4 comprised of a main channel 2, a sub-channel 3, and a merging portion 4 where the main channel 2 and the sub-channel 3 merge together. Panel heaters 8 and 9 are installed in a position such as to be able to heat the interior of the sub-channel 3. The sub-channel 3 and the merging portion are subjected to hydrophobic modification treatment. Water is supplied into the main channel 2, and air is supplied into the sub-channel 3.

### RELATED APPLICATION

This application is a U.S. Continuation Application of International Application PCT/JP2005/002507 filed 10 Feb. 2005.

### TECHNICAL FIELD

The present invention relates to a microchemical system, and in particular relates to a microchemical system that controls the flow of a sample solution in a main channel.

### BACKGROUND ART

To increase the rapidity of chemical reactions or realize reactions with very small amounts of chemicals, on-site analysis or the like, integration technology for carrying out chemical reactions in very small spaces has attracted attention from hitherto, and research has been carried out with vigor throughout the world.

So-called microchemical systems for carrying out mixing, reaction, separation, extraction, detection or the like on a sample solution in a very fine channel are one example of such integration technology for chemical reactions. Examples of reactions carried out in such a microchemical system include diazotization reactions, nitration reactions, and antigen-antibody reactions. Examples of extraction/separation include solvent extraction, electrophoretic separation, and column separation. A microchemical system may be used to provide a single function only, for example for only separation, or may be used to provide a combination of functions.

As an example of a microchemical system for only separation out of the above functions, an electrophoresis apparatus for analyzing extremely small amounts of proteins, nucleic acids or the like has been proposed (see, for example, Japanese Laid-open Patent Publication (Kokai) No. H8-178897). This electrophoresis apparatus has a microchemical system chip (hereinafter referred to merely as a “microchip”) comprised of two glass substrates joined together. Because this chip is plate-shaped, breakage is less likely to occur than in the case of a glass capillary tube having a circular or rectangular cross section, and hence handling is easier.

Moreover, as so-called microvalves for controlling the flow of a sample solution in a channel in such a microchip, ones having structures such as the following have been disclosed.

For example, in first prior art, a micro-stepping motor having a micro-needle attached to a tip thereof is disclosed as a microvalve (see, for example, Keisuke Morishima et al., “Development of Micro Needle-Head Slide Valve Unit for Microfluidic Devices”, 7^{th} International Conference on Miniaturized Chemical and Biochemical Analysis Systems (μ TAS 2003)).

This microvalve is such that the micro-stepping motor 80 is driven so as to raise/lower the micro-needle 81 (FIG. 9A). A channel 83 in a microchip 82 is closed by moving the micro-needle 81 down (FIG. 9B), and is opened by moving the micro-needle 81 up (FIG. 9C).

Moreover, for example, in second prior art, a micro optical switching valve that enables the direction of flow of a sample solution in channels in a microchip to be controlled merely by switching irradiation of light on/off is disclosed as a microvalve (see, for example, Hidenori Nagai et al., “Development of a Micro Optical Switching Valve”, Summary of Presentations at the 8^{th} Meeting on Chemistry and Micro-Nano Systems, P40, Presentation P2-03).

This microvalve is comprised of channels and a He—Cd laser, wherein the channels are made of PDMS, has a T-shaped groove, and is formed by being joined to a quartz substrate coated with titanium oxide.

As shown in FIG. 10, of the formed channels, UV light is irradiated by the He—Cd laser (not shown) from a titanium oxide wall 91 side onto only a channel 92 into which one wishes to make flow a sample solution supplied in from a channel 90, whereby the wall of the channel 92 is made to be super-hydrophilic, so that the sample solution, which is supplied in using a syringe pump or the like, is made to flow into only the channel 92 that has been made to be super-hydrophilic, i.e. and control is carried out such that the sample solution does not flow into a channel 93 that has not been irradiated with UV light.

Furthermore, in third prior art, there is disclosed a method of controlling the flow velocity of a fluid flowing through a channel in a microchip, in which a wall made to be super-hydrophilic using the same method as in the second prior art is formed over a very small range (see, for example, Japanese Laid-open Patent Publication (Kokai) No. 2002-214243).

This method utilizes the characteristic of a liquid crystal panel that the transmissivity to UV light is low when displaying black, and conversely is high when displaying white. Specifically, a liquid crystal panel 101 is placed on a channel 102 in a microchip 100, and the display of the liquid crystal panel 101 is made to be white at a portion 103 thereof corresponding to a portion of the channel 102 where one wishes to increase the hydrophilicity, and the display at the remaining portion 104 of the liquid crystal panel 101 is made to be black. In this state, a UV irradiating apparatus 105 irradiates onto the channel 102 via the liquid crystal panel 101, so that of the channel 102, only the portion irradiated with UV light transmitted through the portion 103 of the liquid crystal panel 101 is made hydrophilic, whereby the flow of a sample 106 through the channel 102 is controlled (see FIG. 11).

However, with the microvalve of the first prior art, when controlling the position of the micro-needle using the micro-stepping motor, there is a risk of the very fine channel in the microchip being damaged. Furthermore, the structure is complex, and hence further size reduction is difficult, and moreover a microchannel especially for applying/reducing pressure required for driving the valve is needed peripherally, and hence there is a problem that this art is not suitable for achieving high integration.

Meanwhile, with the microvalve of the second prior art, there is a problem that in the case that the sample solution is already flowing through a channel in the microchip, the flow of the sample solution cannot be stopped.

Moreover, with the microvalve of the third prior art, because a liquid crystal panel is used, further size reduction is difficult, and moreover electrical circuitry required for driving the liquid crystal panel is needed peripherally, and hence there is a problem that this art is not suitable for achieving high integration. Furthermore, there is also a problem that a sample solution flowing through the channel in the microchip cannot be stopped.

It is an object of the present invention to provide a microchemical system that is capable of controlling the flow of a sample solution flowing through a channel in a microchip.

### DISCLOSURE OF THE INVENTION

To attain the above object, according to a first aspect of the present invention, there is provided a microchemical system comprising a microchip having a channel therein, the microchemical system is characterized in that the channel comprises a main channel through which a liquid having high hydrophilicity is passed, a sub-channel into which is filled a fluid, and a merging portion at which the sub-channel is merged into the main channel, a wall of the main channel having a higher hydrophilicity than each of a wall of the sub-channel and a wall of the merging portion, and the microchemical system has moving means for moving the fluid between the sub-channel and the merging portion.

In the first aspect of the present invention, preferably, the moving means controls movement of the fluid by expanding and contracting the fluid.

Alternatively, preferably, the moving means controls movement of the fluid by pumping the fluid.

Furthermore, preferably, each of the wall of the sub-channel and the wall of the merging portion is subjected to hydrophobic modification treatment.

To attain the above object, according to a second aspect of the present invention, there is provided a microchemical system comprising a microchip having a channel therein, the microchemical system is characterized in that the channel comprises a main channel through which a fluid is passed, a sub-channel into which is filled a liquid having high hydrophilicity, and a merging portion at which the sub-channel is merged into the main channel, a wall of the sub-channel and a wall of the merging portion each having a higher hydrophilicity than a wall of the main channel, and the microchemical system has moving means for moving the liquid between the sub-channel and the merging portion.

In the second aspect of the present invention, preferably, the moving means moves the liquid by pumping the liquid.

Furthermore, preferably, each of the wall of the sub-channel and the wall of the merging portion is subjected to hydrophilic modification treatment.

In the case of the microchemical system according to the first aspect, the liquid does not have compatibility to the fluid.

Furthermore, preferably, a cross sectional area of the sub-channel is less than a cross sectional area of the main channel

Moreover, preferably, the microchemical system has, in the sub-channel, a reservoir portion having a cross sectional area greater than the cross sectional area of the sub-channel.

### BRIEF DESCRIPTION OF THE DRAWINGS

FIG. 1 is a perspective view showing the structure of a microchemical system according to a first embodiment of the present invention;

FIGS. 2A to 2D are views useful in explaining a manufacturing process for a microchip shown in FIG. 1;

FIGS. 3A to 3C are views useful in explaining a microvalve mechanism in the microchemical system shown in FIG. 1;

FIGS. 4A and 4B are views useful in explaining the microvalve mechanism in the microchemical system shown in FIG. 1 following on from FIGS. 3A to 3C;

FIG. 5 is a sectional view showing the structure of a variation of the first embodiment;

FIGS. 6A to 6C are views useful in explaining a microvalve mechanism in a microchemical system according to a second embodiment of the present invention;

FIGS. 7A and 7B are sectional views schematically showing the structure of variations of a sub-channel shown in FIG. 1;

FIG. 8 is a sectional view schematically showing the structure of another variation of the sub-channel shown in FIG. 1;

FIGS. 9A to 9C are views useful in explaining a microvalve mechanism according to first prior art, FIG. 9A being a longitudinal sectional view along a channel, and FIGS. 9B and 9C being cross sectional views across the channel;

FIG. 10 is a view useful in explaining a microvalve mechanism according to second prior art; and

FIG. 11 is a view useful in explaining a method of controlling the flow velocity of a fluid flowing through a channel in a microchip according to third prior art.

### BEST MODE FOR CARRYING OUT THE INVENTION

Embodiments of the present invention will now be described with reference to the drawings.

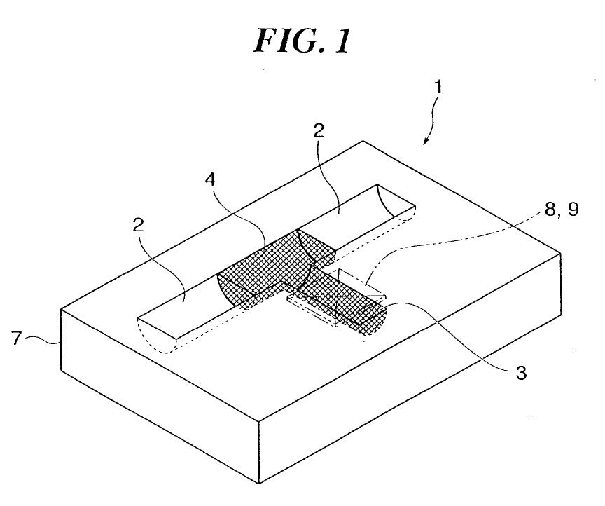

FIG. 1 is a perspective view showing the structure of a microchemical system according to a first embodiment of the present invention.

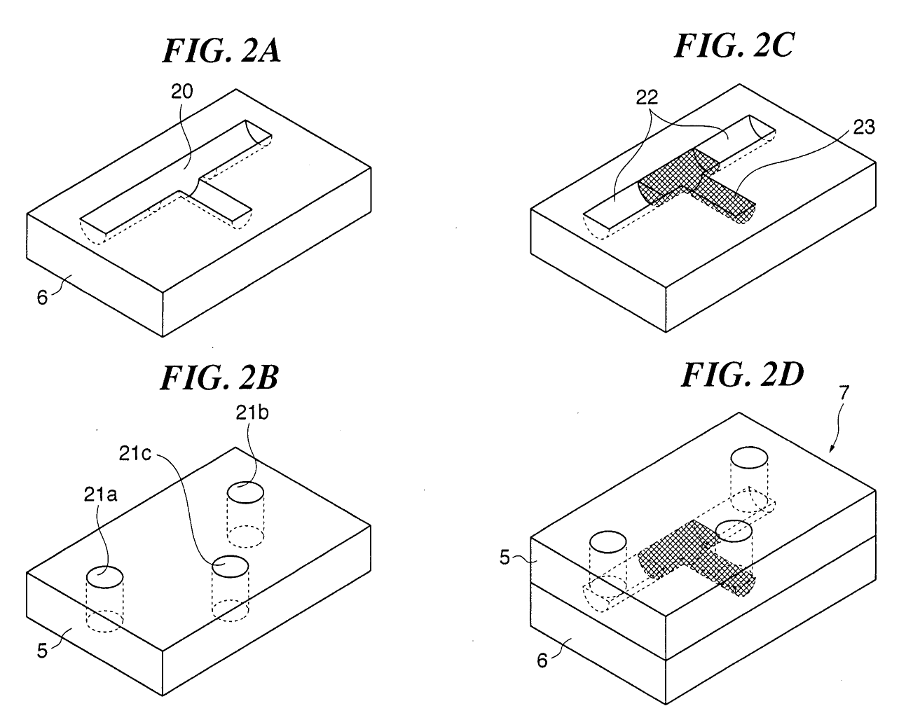

As shown in FIG. 1, the microchemical system 1 is comprised of a microchip 7 having therein a T-shaped channel comprised of a main channel 2 of width 100 μm and depth 50 μm, a sub-channel 3 of width 50 μm and depth 25 μm, and a merging portion 4 that is part of the main channel 2 and constitutes a portion where the main channel 2 and the sub-channel 3 merge together, and panel heaters 8 and 9 installed in a position such as to be able to heat the interior of the sub-channel 3. Moreover, the microchip 7 is connected to through holes 21a, 21b, and 21c shown in FIGS. 2B and 2D, described below, for supplying in/discharging a sample solution or the like from ends of the main channel 2 and the sub-channel 3, but these through holes 21a, 21b, and 21c are omitted from FIG. 1.

The microchip 7 is made of glass. Any glass such as a soda lime glass, an aluminoborosilicate glass, an aluminosilicate glass, an alkali-free glass, or a quartz glass may be used, but it is preferable to use a quartz glass, which has the highest hydrophilicity.

FIGS. 2A to 2D are views useful in explaining a manufacturing process for the microchip shown in FIG. 1.

First, a groove 20 that will become channels constituting the main channel 2 and the sub-channel 3 is formed either all in one or else in individual sections in a wall of a plate-shaped substrate 6 using a fluorine etching method (FIG. 2A), and the through holes 21a, 21b, and 21c for supplying/discharging a sample solution or the like into/out of the channels in the microchip 7 are formed in a plate-shaped substrate 5 by hole forming processing using a drill (FIG. 2B).

A masking agent is applied onto portions 22 of a wall of the groove 20 formed in the plate-shaped substrate 6 that will become the main channel 2 shown in FIG. 1, and then an organic siloxane such as polydimethylsiloxane (PDMS) is applied over the whole of the wall of the groove 20, and heating is carried out to bring about polymerization, and then the applied masking agent is removed. As a result, hydrophobic modification treatment is carried out on a portion 23 of the wall of the groove 20 that will become the sub-channel 3 and the merging portion 4 shown in FIG. 1 (FIG. 2C). Alternatively, a fluorinated organosilane compound such as a perfluoroalkylsilane may be used for the hydrophobic modification treatment. As a result of the treatment, whereas the contact angle of water at the portions 22 is not more than 20°, the contact angle of water at the portion 23 becomes not less than 70°, which indicates that the hydrophilicity of the latter portion is reduced, i.e. the hydrophobicity thereof is increased.

Finally, the plate-shaped substrate 5 having the through holes 21a, 21b, and 21c formed therein is bonded on so as to cover the groove 20 in the plate-shaped substrate 6, thus manufacturing the microchip 7 (FIG. 2D).

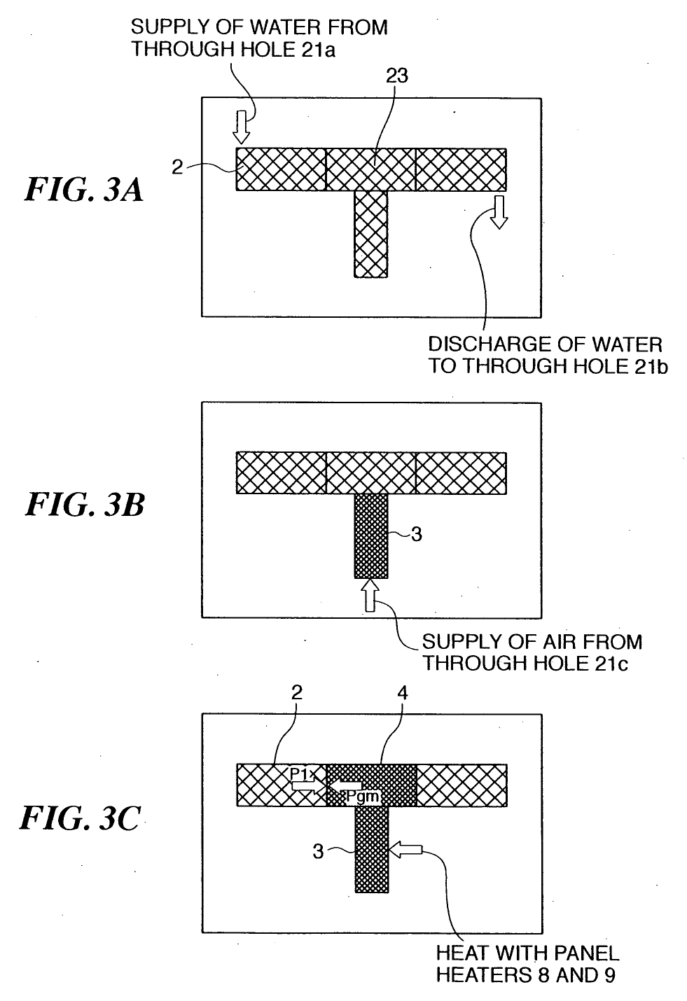

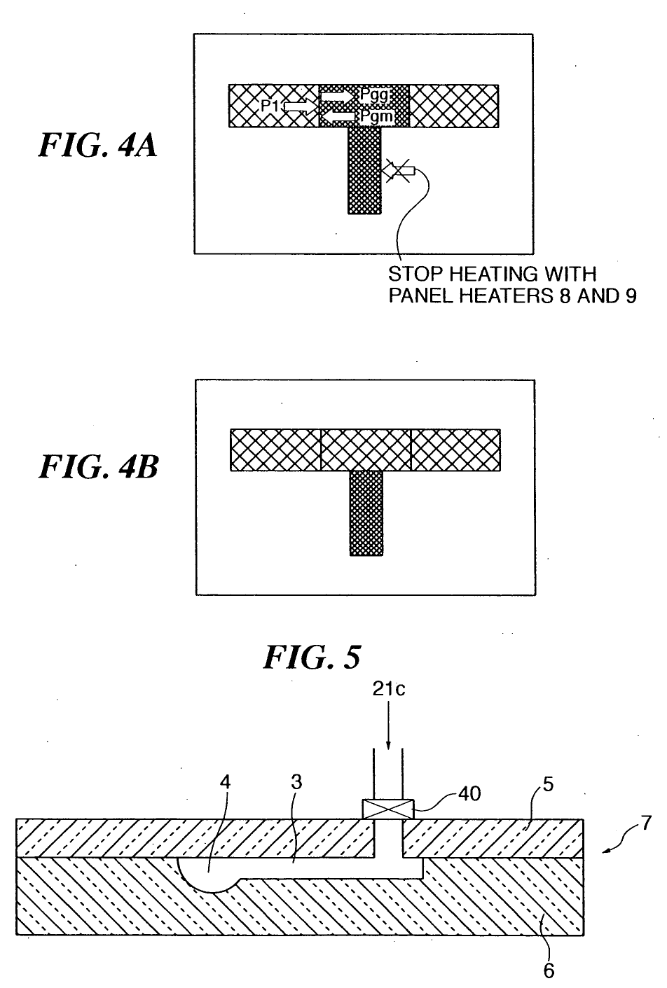

FIGS. 3A to 3C and FIGS. 4A and 4B are views useful in explaining a microvalve mechanism in the microchemical system shown in FIG. 1.

In a state in which water is being supplied into the main channel 2 from the through hole 21a and the supplied water is being discharged to the through hole 21b from the main channel 2 (FIG. 3A), air is supplied in from the through hole 21c so as to fill the sub-channel 3 (FIG. 3B).

Next, the sub-channel 3 is heated using the panel heaters 8 and 9, thus expanding the volume of the air in the sub-channel 3, so that air is introduced in as far as the merging portion 4 (FIG. 3C). As a result, a gas-liquid interface arises in the main channel 2, and a pressure Pgm arises that acts to make the water in the main channel 2 stay at a boundary between the portion 23 that has been subjected to the hydrophobic modification treatment and the portion 22 having high hydrophilicity shown in FIG. 2C. This pressure acts as a microvalve stopping the flow of the water that was flowing through the main channel 2.

This phenomenon arises due to so-called surface tension whereby a hydrophilic liquid such as water having a high wettability acts to broaden the contact with the interface at the portion 22 having high hydrophilicity, and on the other hand acts to reduce the contact with the interface at the portion 23 having high hydrophobicity.

Note, however, that in the case that the pressure P1 of the water flowing through the main channel 2 is higher than the above pressure Pgm, the flow of the water flowing through the main channel 2 cannot be stopped. The pressure when supplying the water into the main channel 2 from the through hole 21a must thus be controlled to be not more than a predetermined value.

Subsequently, upon the heating by the panel heaters 8 and 9 shown in FIG. 1 being stopped, the thermally expanded air is cooled so that the volume thereof contracts, and a pressure Pgg acting to return the air into the sub-channel 3 arises (FIG. 4A). Once the pressure Pgg becomes greater than the pressure Pgm acting to stay at the gas-liquid interface, the air returns into the sub-channel 3, and hence the water in the main channel 2 starts to flow again (FIG. 4B).

In the present embodiment, the cross sectional area of the sub-channel 3 is preferably less than the cross sectional area of the main channel 2. This is because then it becomes easy to control the value of the pressure Pgg.

Moreover, in the present embodiment, the fluid supplied into the main channel 2 is water, but so long as the liquid has high hydrophilicity, such as alcohols, there is no limitation to this.

Meanwhile, in the present embodiment, the fluid supplied into the sub-channel 3 is air, but so long as this fluid does not have compatibility to the fluid flowing through the main channel 2, there is no limitation to this, but rather another gas or a liquid may be used. As a result, when the fluid supplied into the sub-channel 3 has been moved into the merging portion 4, the fluid in the main channel 2 can be prevented from dissolving in the merging portion 4.

In the case of supplying a liquid into the sub-channel 3, examples of such liquid include hydrophobic organic solvents, specifically benzene, toluene, and kerosene. In this case, unlike for air, the volume change upon heating is not large for the liquid, and hence instead of the panel heaters 8 and 9, it is preferable to install bellows 40 at one end of the sub-channel 3, and after the organic solvent has been introduced into the merging portion 4, to return the organic solvent into the sub-channel 3 by pumping using the bellows 40 (see FIG. 5).

A microchemical system according to a second embodiment of the present invention will now be described.

A microchip used in the microchemical system according to the second embodiment differs from the first embodiment in that the microchip is made of an acrylic resin rather than glass, and the sub-channel and the merging portion provided in part of the main channel are (i.e. the portion 23 in FIG. 2C is) subjected to hydrophilic modification treatment rather than hydrophobic modification treatment. Other than this, the microchip has basically the same structure as the microchip used in the microchemical system according to the first embodiment (FIGS. 1 to 5).

In the present embodiment, the hydrophilic modification treatment carried out on the portion 23 is carried out by first coating the portion 23 with a titanium oxide thin film by mask deposition using a sputtering method or the like, and then irradiating with UV light.

Moreover, the contact angle of water on the acrylic resin is generally approximately 50°, and hence to increase the hydrophobicity at the portions 22 of the microchip 7, hydrophobic modification treatment like that in the first embodiment (FIG. 2C) may be carried out.

In the present embodiment, the microchip 7 is made of an acrylic resin, but so long as the material is hydrophobic, there is no limitation to this. For example, any of polyethylene, polypropylene, a polycarbonate, or the like may be used instead.

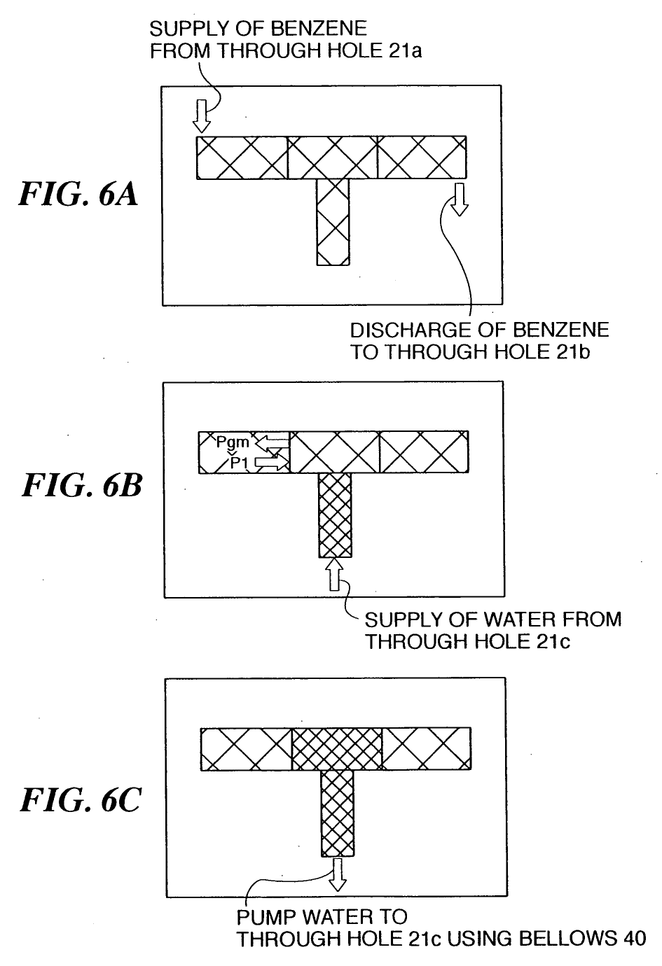

FIGS. 6A to 6C are views useful in explaining a microvalve mechanism in the microchemical system according to the present embodiment.

In a state in which benzene is being supplied into the main channel 2 from the through hole 21a and the benzene is being discharged to the through hole 21b from the main channel 2 (FIG. 6A), water is supplied in from the through hole 21c so as to fill the sub-channel 3 and the merging portion 4 (FIG. 6B).

A pressure Pgm acting to make the water-benzene interface stay at the boundary between the portion 23 and the portion 22 arises as in the first embodiment, and acts as a microvalve stopping the flow of the benzene that was flowing through the main channel 2.

Subsequently, upon the water in the merging portion 4 being released into the sub-channel 3 using the bellows 40 (FIG. 6C), the benzene in the main channel 2 starts to flow again.

In the present embodiment, the liquid supplied into the sub-channel 3 is water, but so long as the liquid has high hydrophilicity, such as alcohols, there is no limitation to this.

Meanwhile, in the present embodiment, the fluid supplied into the main channel 2 is benzene, but so long as this fluid does not have compatibility to the liquid flowing through the sub-channel 3, there is no limitation to this, but rather another fluid may be used. As a result, when the liquid supplied into the sub-channel 3 has been moved into the merging portion 4, the fluid in the main channel 2 can be prevented from dissolving in the merging portion 4.

In the case of supplying another liquid into the main channel 2, examples include hydrophobic organic solvents, specifically toluene and kerosene. As a result, when the liquid supplied into the sub-channel 3 has been moved into the merging portion 4, the fluid in the main channel 2 can be prevented from dissolving in the merging portion 4.

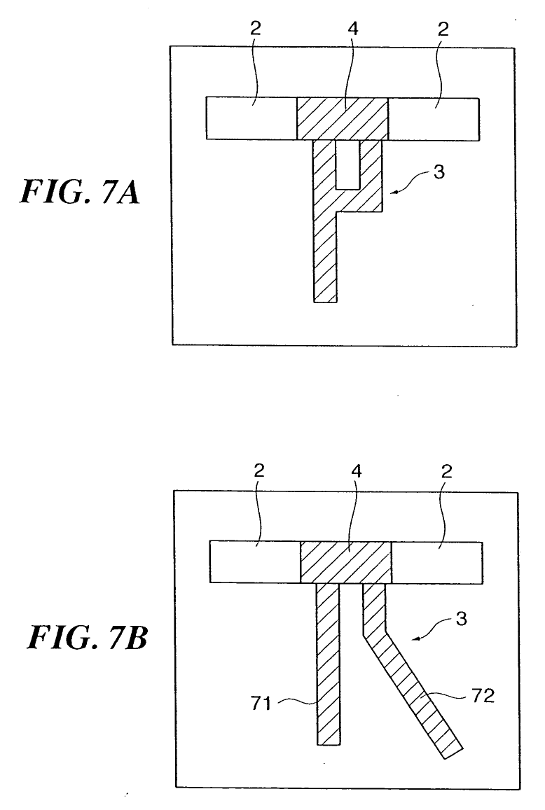

In the microchip 7 described above, the sub-channel 3 is constituted from a single channel, but the sub-channel 3 may instead be a branched channel having merging openings on the upstream side and the downstream side in the merging portion 4 as shown in FIG. 7A, or may be comprised of a fluid supply channel 71 through which the fluid is supplied into the merging portion 4 and a fluid discharge channel 72 through which the fluid supplied into the merging portion 4 is discharged out from the merging portion 4 as shown in FIG. 7B.

Furthermore, there may be a reservoir portion (not shown) between the sub-channel 3 and the through hole 21c, having a cross sectional area greater than the cross sectional area of the sub-channel 3. As a result, control of the movement of the fluid flowing through the sub-channel 3 (air in the first embodiment, water in the second embodiment, etc.) can be carried out reliably.

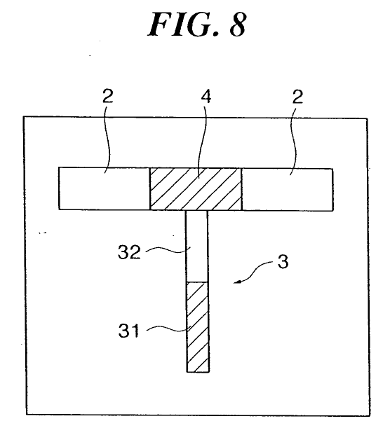

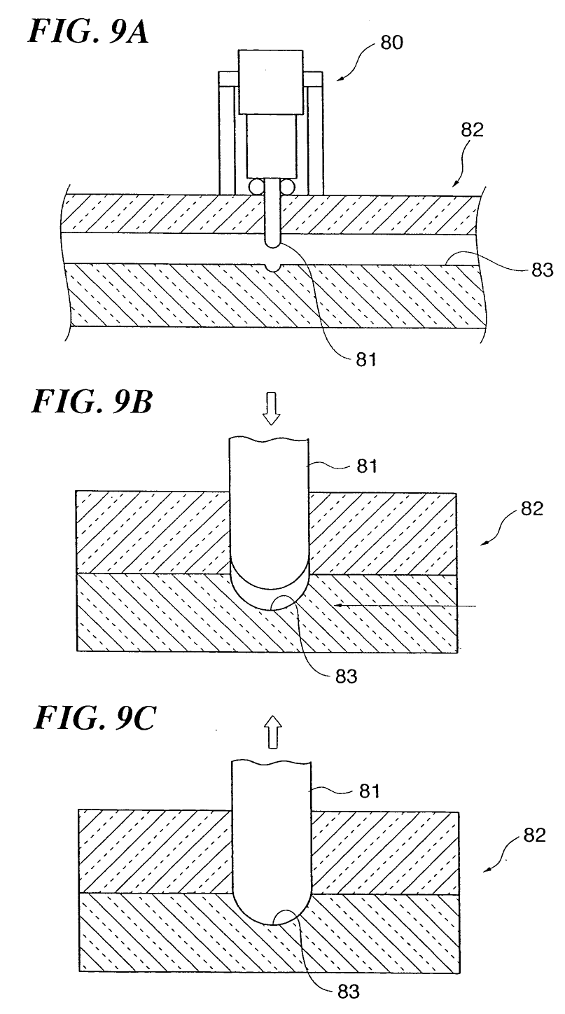

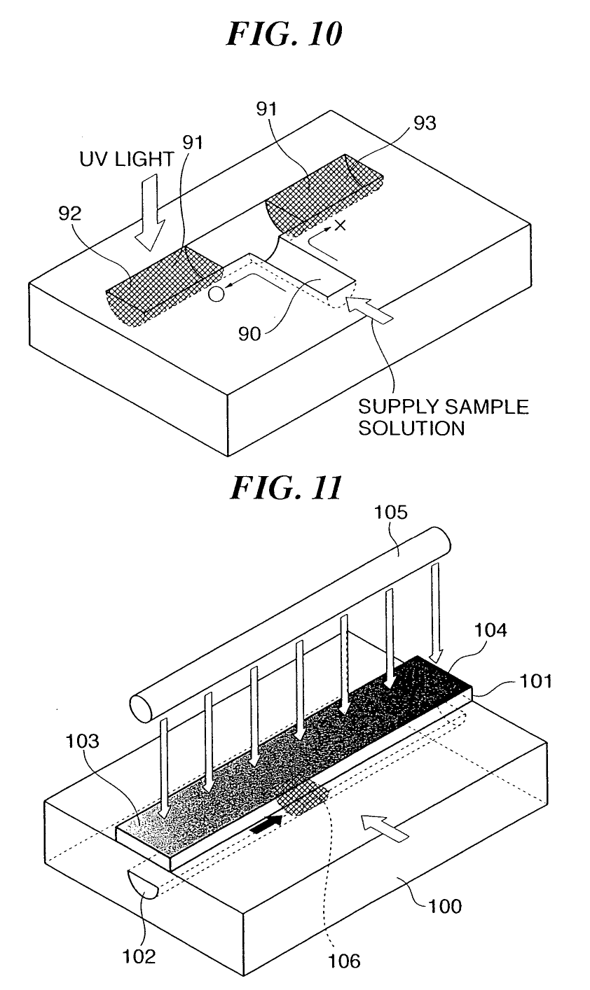

Moreover, as shown in FIG. 8, a portion 32 of the sub-channel 3 on the merging portion 4 side may be made to have a hydrophilic wall as for the main channel 2, the remaining portion 31 of the sub-channel 3 being made to have a hydrophobic wall. As a result, when gas (air etc.) that has been acting as a valve stopping the fluid in the main channel 2 is returned into the sub-channel 3 so as to put the valve into an open state, the gas-liquid interface in the sub-channel 3 tries to stay at the interface between the portion 31 and the portion 32, whereby control of the movement of the fluid flowing through the sub-channel 3 can be carried out more reliably.

### INDUSTRIAL APPLICABILITY

According to a microchemical system of the present invention, the wall of a main channel in a microchip through which a liquid having high hydrophilicity is passed has a higher hydrophilicity than each of the wall of a sub-channel and the wall of a merging portion, and a fluid is moved between the sub-channel and the merging portion. As a result, surface tension that arises at the interface between the liquid having high hydrophilicity and the fluid acts as a microvalve stopping the flow of the liquid that was flowing through the main channel. The flow of a sample solution flowing through the channel in the microchip can thus be controlled.

According to a microchemical system of the present invention, movement of the fluid is controlled by expanding and contracting the fluid. As a result, the flow of the sample solution flowing through the channel in the microchip can be controlled reliably, and moreover high integration can be achieved for the microchemical system.

According to a microchemical system of the present invention, movement of the fluid is controlled by pumping the fluid. As a result, the flow of the sample solution flowing through the channel in the microchip can be controlled reliably.

According to a microchemical system of the present invention, each of the wall of the sub-channel and the wall of the merging portion is subjected to hydrophobic modification treatment. As a result, the microchemical system can be manufactured simply and reliably.

According to a microchemical system of the present invention, the wall of a sub-channel into which is filled a liquid having high hydrophilicity and the wall of a merging portion each have a higher hydrophilicity than the wall of a main channel, and the liquid passed into the sub-channel is moved between the sub-channel and the merging portion. As a result, surface tension that arises at the interface between the liquid having high hydrophilicity and the fluid acts as a microvalve stopping the flow of a fluid that was flowing through the sub-channel. The flow of a sample solution flowing through the channel in the microchip can thus be controlled.

According to a microchemical system of the present invention, the liquid having high hydrophilicity in the sub-channel is moved by pumping the liquid. As a result, the flow of the sample solution flowing through the channel in the microchip can be controlled reliably.

According to a microchemical system of the present invention, each of the wall of the sub-channel and the wall of the merging portion is subjected to hydrophilic modification treatment. As a result, the microchemical system can be manufactured simply and reliably

According to a microchemical system of the present invention, the liquid having high hydrophilicity does not have compatibility to the fluid. As a result, there is no dissolving and mixing of the liquid and the fluid with one another in the merging portion, and hence the flow in the main channel can be stopped reliably.

According to a microchemical system of the present invention, the cross sectional area of the sub-channel is less than the cross sectional area of the main channel. As a result, the pressure of the fluid or the like filled into the sub-channel can be controlled easily.

According to a microchemical system of the present invention, in the sub-channel, there is a reservoir portion having a cross sectional area greater than the cross sectional area of the sub-channel. As a result, movement of the fluid flowing through the sub-channel, for example the liquid having high hydrophilicity, can be controlled reliably.

1. A microchemical system comprising a microchip having a channel therein, the microchemical system characterized in that:
said channel comprises a main channel through which a liquid having high hydrophilicity is passed, a sub-channel into which is filled a fluid, and a merging portion at which said sub-channel is merged into said main channel, a wall of said main channel having a higher hydrophilicity than each of a wall of said sub-channel and a wall of said merging portion; and
the microchemical system has moving means for moving the fluid between said sub-channel and said merging portion.

2. A microchemical system as claimed in claim 1, characterized in that aid moving means controls movement of the fluid by expanding and contracting the fluid.

3. A microchemical system as claimed in claim 1, characterized in that said moving means controls movement of the fluid by pumping the fluid.

4. A microchemical system as claimed in claim 1, characterized in that each of the wall of said sub-channel and the wall of said merging portion is subjected to hydrophobic modification treatment.

5. A microchemical system comprising a microchip having a channel therein, the microchemical system characterized in that:
said channel comprises a main channel through which a fluid is passed, a sub-channel into which is filled a liquid having high hydrophilicity, and a merging portion at which said sub-channel is merged into said main channel, a wall of said sub-channel and a wall of said merging portion each having a higher hydrophilicity than a wall of said main channel; and
the microchemical system has moving means for moving the liquid between said sub-channel and said merging portion.

6. A microchemical system as claimed in claim 5, characterized in that said moving means moves the liquid by pumping the liquid.

7. A microchemical system as claimed in claim 5, characterized in that each of the wall of said sub-channel and the wall of said merging portion is subjected to hydrophilic modification treatment.

8. A microchemical system as claimed in claim 1, characterized in that the liquid does not have compatibility to the fluid.

9. A microchemical system as claimed in claim 1, characterized in that a cross sectional area of said sub-channel is less than a cross sectional area of said main channel.

10. A microchemical system as claimed in claim 9, characterized by having, in said sub-channel, a reservoir portion having a cross sectional area greater than the cross sectional area of said sub-channel.
